(19) United States
(12) Patent Application Publication
(10) Pub. No.: US 2026/0258506 A1
(43) Pub. Date: Sep. 3, 2026

(54) MARKER USED TO DETECT INVASIVE BLADDER CANCER, AND APPLICATION THEREOF

(71) Applicant: Lisen Imprinting Diagnostics Wuxi Co., Ltd., Wuxi (CN)

(72) Inventors: Tong Cheng, Wuxi (CN); Ning Zhou, Wuxi (CN)

(21) Appl. No.: 19/537,284

(22) Filed: Feb. 11, 2026

Related U.S. Application Data
(63) Continuation of application No. 17/295,629, filed on Nov. 18, 2019, now abandoned.

(30) Foreign Application Priority Data
Nov. 21, 2018 (CN) 201811391567.0

(51) Int. Cl. C12Q 1/6886

(52) U.S. Cl. CPC C12Q 1/6886; C12Q 2600/112; C12Q 2600/158

(57) ABSTRACT

Provided in the present application are a marker used to detect invasive bladder cancer and an application thereof, the marker being imprinted gene Grb10 and/or imprinted gene Diras3. The marker of the present application can accurately detect the invasiveness of a bladder cancer, thereby providing guidance on the choice of the surgical method to be used on the bladder cancer, reducing the chance of post-operative recurrence and metastasis. The detection method of the present application is different from its immunohistochemical counterparts in that it can reduce false positives and other negative effects. Moreover, targeting drugs or methods that are found to work at the loss-of-imprinting-affected sites of bladder-cancer-invasiveness-related imprinted genes to silence, delete, or rearrange those genes can be used to guide subsequent treatment and medication.

### TECHNICAL FIELD

The present application relates to the field of biotechnology, to the field of genetic diagnosis, to a marker for tumor detection, and more particularly to a marker used to detect invasive bladder cancer and an application thereof.

### DESCRIPTION OF RELATED ART

Bladder cancer is the most common malignant tumor in the urinary system and has the highest morbidity of all the malignant tumors in the urinary system. According to statistics of the World Health Organization (WHO), or more specifically World Cancer Report 2014, the year 2012 saw 429,793 new cases of bladder cancer worldwide, including 165,084 deaths. In China, the same year saw 55,486 newly diagnosed cases of bladder cancer, including 26,820 deaths. Clinically, 85%-90% of newly diagnosed bladder cancer cases are non-muscle-invasive bladder cancer, which despite a recurrence rate as high as 75% after the first resection, seldom grows into the muscle and rarely spreads to other parts of the body. The remaining 10%-15%, however, are muscle-invasive bladder cancer, which will eventually become metastatic and fatal. Generally, non-muscle-invasive bladder cancer has a relatively high survival rate if cystoscopic examination is carried out on a regular basis to remove any recurring tumor, via the cystoscope, in a timely manner. Muscle-invasive bladder cancer requires a larger portion of the bladder to be cut off or even the entire bladder removed, and necessitates chemical therapy in order to produce a better treatment result. Therefore, early detection as to whether a bladder cancer will grow into the muscle is of great importance regarding the choice of the treating method and helps save the patient's life.

Genomic imprinting is a gene regulation method in epigenetics and is characterized by methylating an allele from a specific parent such that only one allele of the corresponding gene is expressed while the other allele is in a silenced state. A gene regulated in this way is referred to as an imprinted gene. Loss of imprinting is an epigenetic change in which the silenced allele of an imprinted gene is demethylated and is thus activated and expressed. Numerous studies have shown that loss of imprinting exists widely in all kinds of cancers and takes place earlier than morphological changes in cells and tissue. Loss of imprinting, however, seldom occurs in healthy cells, which is in stark contrast to the case with cancer cells. Therefore, the methylated state of an imprinted gene can serve as a pathological marker and used in conjunction with specific molecular detection techniques to analyze cellular abnormality.

As the functions of an imprinted gene cover cell signaling, cell cycle regulation, substance transport across the cell membrane, the formation of extracellular matrix, and so on, the expressed functions and expressed quantity of an imprinted gene in a certain cancer may differ from those in another cancer, with the difference in expressed quantity being huge. An imprinted gene, therefore, may have different sensitivity and specificity to a certain cancer from another imprinted gene. The differences in sensitivity and specificity have a huge impact on the invasion and metastasis of tumor cells during tumor development and on the prognosis of tumors.

According to the above, no diagnostic markers are currently available to clearly distinguish an invasive bladder cancer from a non-invasive one. That is to say, there is presently no way to analyze bladder-cancer-invasiveness-related changes in a molecular marker on a cellular level in order to provide more accurate prognosis and diagnosis information.

### BRIEF SUMMARY OF THE INVENTION

In view of the deficiency of the prior art and in order to meet practical needs, the present application provides a marker for detecting invasive bladder cancer and an application thereof so that by detecting an imprinted gene, an invasive bladder cancer can be distinguished from a non-invasive one, thereby providing a basis for subsequent treatment.

To achieve the foregoing objective, the following technical solution is used:

According to the first aspect of the present application, a marker for invasive bladder cancer is provided, wherein the marker is imprinted gene Grb10 and/or imprinted gene Diras3.

In the present application, the following expressed quantities of imprinted gene Grb10 and of imprinted gene Diras3 are calculated: a total expressed quantity of each imprinted gene, an expressed quantity of each imprinted gene with a loss of imprinting, and an expressed quantity of each imprinted gene with a copy number variation. The ratio of the expressed quantity of imprinted gene Grb10 with a copy number variation to the expressed quantity of imprinted gene Diras3 with a copy number variation is then used as a bladder infiltration factor (BIF) by which to determine the invasiveness of a bladder cancer. The higher the infiltration factor, the more invasive the bladder cancer.

According to the present application, the following formulas are used to calculate the total expressed quantity of an imprinted gene, the expressed quantity of the imprinted gene being normal, the expressed quantity of the imprinted gene with a loss of imprinting, and the expressed quantity of the imprinted gene with a copy number variation:
total expressed quantity of the imprinted gene=(b+c+d)/(a+b+c+d)×100%;
expressed quantity of the imprinted gene being normal=b/(b+c+d)×100%;
expressed quantity of the imprinted gene with a loss of imprinting=c/(b+c+d)×100%; and
expressed quantity of the imprinted gene with a copy number variation=d/(b+c+d)×100%; where a is the number of cell nuclei that, after the corresponding cells are stained with hematoxylin, show no mark in each cell nucleus, meaning the imprinted gene is not expressed in those cell nuclei; b is the number of cell nuclei that, after the corresponding cells are stained with hematoxylin, show one red/brown mark in each cell nucleus, meaning the imprinted gene is present in those cell nuclei; c is the number of cell nuclei that, after the corresponding cells are stained with hematoxylin, show two red/brown marks in each cell nucleus, meaning the imprinted gene is affected by a loss of imprinting in those cell nuclei; and d is the number of cell nuclei that, after the corresponding cells are stained with hematoxylin, show more than two red/brown marks in each cell nucleus, meaning the imprinted gene shows a copy number variation in those cell nuclei.

According to the present application, when the total expressed quantity of imprinted gene Diras3 is less than 8% or the expressed quantity of imprinted gene Diras3 with a copy number variation is not greater than 1%, the bladder cancer in question is determined to have non-obvious invasiveness if the total expressed quantity of imprinted gene Grb10 is less than 8%, or if the expressed quantity of imprinted gene Grb10 with a copy number variation is not greater than 1.5% and the infiltration factor is not greater than 1.5, wherein the infiltration factor is the ratio of the expressed quantity of imprinted gene Grb10 with a copy number variation to the expressed quantity of imprinted gene Diras3 with a copy number variation.

In the present application, a bladder cancer having non-obvious invasiveness refers to a condition in which whether the bladder cancer is invasive cannot be clearly determined, and in which it is required to allow the bladder cancer to grow in order to carry out further detection.

According to the present application, when the total expressed quantity of imprinted gene Diras3 is not less than 8% and the expressed quantity of imprinted gene Diras3 with a copy number variation is not greater than 1%, the bladder cancer in question is determined to be a mixed to invasive bladder cancer if the total expressed quantity of imprinted gene Grb10 is not less than 8%, the expressed quantity of imprinted gene Grb10 with a copy number variation is not greater than 1.5%, and the infiltration factor is greater than 1.5, wherein the infiltration factor is the ratio of the expressed quantity of imprinted gene Grb10 with a copy number variation to the expressed quantity of imprinted gene Diras3 with a copy number variation.

In the present application, a mixed to invasive bladder cancer refers to a bladder cancer that includes both invasive bladder cancer and non-invasive bladder cancer, in which the invasive bladder cancer grows faster than the non-invasive bladder cancer.

According to the present application, when the total expressed quantity of imprinted gene Diras3 is not less than 8% and the expressed quantity of imprinted gene Diras3 with a copy number variation is greater than 1%, the bladder cancer in question is determined to be a mixed bladder cancer if the total expressed quantity of imprinted gene Grb10 is not less than 8%, the expressed quantity of imprinted gene Grb10 with a copy number variation is greater than 1.5%, and the infiltration factor is not greater than 1.5, or if the total expressed quantity of imprinted gene Grb10 is not less than 8%, the expressed quantity of imprinted gene Grb10 with a copy number variation is not greater than 1.5%, and the infiltration factor is not less than 1.5, wherein the infiltration factor is the ratio of the expressed quantity of imprinted gene Grb10 with a copy number variation to the expressed quantity of imprinted gene Diras3 with a copy number variation.

In the present application, a mixed bladder cancer refers to a bladder cancer that includes both invasive bladder cancer and non-invasive bladder cancer, and that requires further detection as the bladder cancer develops.

According to the present application, when the total expressed quantity of imprinted gene Diras3 is less than 8% or the expressed quantity of imprinted gene Diras3 with a copy number variation is not greater than 1%, the bladder cancer in question is determined to be invasive if the total expressed quantity of imprinted gene Grb10 is not less than 8% and the expressed quantity of imprinted gene Grb10 with a copy number variation is greater than 1.5%.

According to the present application, when the total expressed quantity of imprinted gene Diras3 is not less than 8% and the expressed quantity of imprinted gene Diras3 with a copy number variation is greater than 1%, the bladder cancer in question is determined to be non-invasive if the total expressed quantity of imprinted gene Grb10 is less than 8%, or if the total expressed quantity of imprinted gene Grb10 is not less than 8%, the expressed quantity of imprinted gene Grb10 with a copy number variation is not greater than 1.5%, and the infiltration factor is less than 1.5, wherein the infiltration factor is the ratio of the expressed quantity of imprinted gene Grb10 with a copy number variation to the expressed quantity of imprinted gene Diras3 with a copy number variation.

According to the present application, when the total expressed quantity of imprinted gene Diras3 is not less than 8% and the expressed quantity of imprinted gene Diras3 with a copy number variation is greater than 1%, the bladder cancer in question is determined to be invasive if the total expressed quantity of imprinted gene Grb10 is not less than 8%, the expressed quantity of imprinted gene Grb10 with a copy number variation is greater than 1.5%, and the infiltration factor is greater than 1.5, wherein the infiltration factor is the ratio of the expressed quantity of imprinted gene Grb10 with a copy number variation to the expressed quantity of imprinted gene Diras3 with a copy number variation.

The aforesaid criteria of the present application for determining the invasiveness of a bladder cancer can be summarized as follows:

where Z15 is imprinted gene Diras3, and Z11 is imprinted gene Grb10.

As would be understood by a person skilled in the art, the foregoing criteria of the present application may fluctuate as the number of samples increases. The relationship between the total expressed quantity of the marker, the expressed quantity of the marker with a copy number variation, and an invasive bladder cancer may fluctuate within +15% of the values stated above without departing from the scope of the present application.

According to the present application, imprinted gene Grb10 is expressed in an invasive bladder cancer cell line, but imprinted gene Diras3 is not expressed in the invasive bladder cancer cell line.

According to the present application, the invasive bladder cancer cell line is any one, or a combination of at least two, of the T24 cell line, the J82 cell line, and the UMUC3 cell line.

According to the present application, imprinted gene Grb10 is not expressed in a non-invasive bladder cancer cell line, but imprinted gene Diras3 is expressed in the non-invasive bladder cancer cell line.

According to the present application, the non-invasive bladder cancer cell line is the 5637 cell line.

The detection method of the present application is different from immunohistochemical methods, DNA sequencing methods, DNA methylation analysis methods, and fluorescence in situ hybridization (FISH) methods in that it can reduce false positives and other negative effects.

According to another aspect of the present application, a use of the marker described in relation to the first aspect of the present application in preparing a drug or reagent for diagnosing bladder cancer is provided.

Compared with the prior art, the present application has the following advantageous effects: (1) The marker of the present application can accurately detect the invasiveness of a bladder cancer, thereby providing guidance on the choice of the surgical method to be used on the bladder cancer, reducing the chance of post-operative recurrence and metastasis.

(2) The samples required for use according to the present application are readily available and may be biopsy cells obtained through a cystoscope or detached cells in urine. The marker of the present application allows an invasive bladder cancer to be distinguished from a non-invasive one in an early stage and is therefore useful in early general surveys and cancer patients' post-operative follow-ups, in particular when cancer recurrence is suspected. The marker can shorten the time required for diagnosis and thus contributes greatly to saving human lives.

(3) The detection method of the present application is different from its immunohistochemical counterparts in that it can reduce false positives and other negative effects. Moreover, targeting drugs or methods that are found to work at the loss-of-imprinting-affected sites of bladder-cancer-invasiveness-related imprinted genes to silence, delete, or rearrange those genes can be used to guide subsequent treatment and medication.

### BRIEF DESCRIPTION OF THE SEVERAL VIEWS OF THE DRAWINGS

FIG. 1 shows the detection results, or more particularly the bladder infiltration factors, of different bladder cancer cell lines; and

FIG. 2 shows the detection results, or more particularly the bladder infiltration factors, of 17 bladder cancer samples.

### DETAILED DESCRIPTION OF THE INVENTION

To further explain the technical solution used in the present application and its effects, certain embodiments are detailed below with reference to the accompanying drawings to expound the technical solution of the present application. The present application, however, is not limited to the embodiments described below.

### Embodiment 1: Detection of the Invasiveness of Bladder Cancer Cell Lines

The imprinted gene detection method used in this embodiment includes the following steps:
(1) Cultured bladder cancer cell lines 5637, UMUC3, T24, and J82 were placed in 10% neutral buffered formalin in order to be fixed, lest the RNA degrade. After fixing for 24 hours, the cell lines were loaded onto positively charged slides. The slides were then baked in a 37° C. oven for at least 3 hours.
(2) The sample processing methods of RNAscope were used to block the activity of the endogenous peroxidase in the samples, to permeabilize the samples, and to expose the RNA molecules.
(3) Probe design: Specific primers, or probes, were designed according to the sequences of the imprinted genes to be detected.

The probes were designed according to the to-be-detected imprinted genes Grb10 and Diras3. More specifically, a sequence was selected from the intron of each of the aforesaid genes as the corresponding probe. The probes were designed by Advanced Cell Diagnostics.
(4) RNAscope reagent kits were used to perform in-situ hybridization between the probes in step (3) and the test samples.
(5) After staining with hematoxylin for signal amplification, the expression of the imprinted genes was analyzed via microscopic imaging.

The total expressed quantity of each imprinted gene, the expressed quantity of each imprinted gene being normal, the expressed quantity of each imprinted gene with a loss of imprinting, and the expressed quantity of each imprinted gene with a copy number variation were calculated using the following formulas:
total expressed quantity of an imprinted gene=(b+c+d)/(a+b+c+d)×100%;
expressed quantity of the imprinted gene being normal=b/(b+c+d)×100%;
expressed quantity of the imprinted gene with a loss of imprinting (LOI)=c/(b+c+d)×100%; and
expressed quantity of the imprinted gene with a copy number variation (CNV)=d/(b+c+d)×100%;
   where a is the number of cell nuclei that, after the corresponding cells are stained with hematoxylin, show no mark in each cell nucleus, meaning the imprinted gene is not expressed in those cell nuclei; b is the number of cell nuclei that, after the corresponding cells are stained with hematoxylin, show one red/brown mark in each cell nucleus, meaning the imprinted gene is present in those cell nuclei; c is the number of cell nuclei that, after the corresponding cells are stained with hematoxylin, show two red/brown marks in each cell nucleus, meaning the imprinted gene is affected by a loss of imprinting in those cell nuclei; and d is the number of cell nuclei that, after the corresponding cells are stained with hematoxylin, show more than two red/brown marks in each cell nucleus, meaning the imprinted gene shows a copy number variation in those cell nuclei.

After that, the bladder infiltration factors were calculated. Each bladder infiltration factor was the ratio of the corresponding expressed quantity of imprinted gene Grb10 with a copy number variation to the corresponding expressed quantity of imprinted gene Diras3 with a copy number variation.

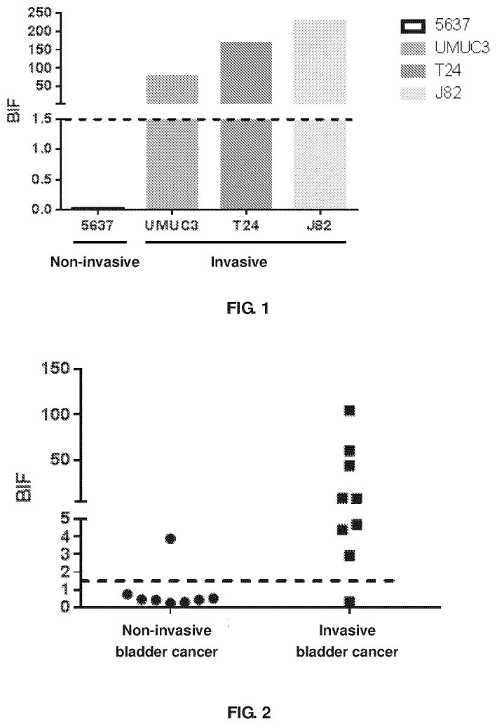

As can be seen in FIG. 1, the bladder infiltration factor of the non-invasive bladder cancer cell line 5637 is less than 1.5, and the bladder infiltration factors of the invasive bladder cancer cell lines UMUC3, T24, and J82 are all greater than 1.5.

Embodiment 2: Detection of the Invasiveness of 17 Bladder Cancer Samples 17 tissue samples, including biopsy samples obtained through cystoscopy, were obtained from bladder cancer patients and were processed as follows.
(1) Bladder cancer tissue or cells were obtained and placed in 10% neutral buffered formalin in order to be fixed, lest the RNA degrade. After fixing for 24 hours and embedment in paraffin (FFPE), the embedded tissue or cells were cut into 10 μm-thick sections, which were loaded onto positively charged slides. The slides were then baked in a 40° C. oven for at least 3 hours.
(2) The sample processing methods of RNAscope were used to dewax the sections, block the activity of the endogenous peroxidase in the samples, permeabilize the tissue or cells, and expose the RNA molecules.

The remaining steps of the detection method were the same as those in embodiment 1.

As can be seen in FIG. 2, the bladder infiltration factors of the majority of the non-invasive bladder cancer samples are less than 1.5, and the bladder infiltration factors of the majority of the invasive bladder cancer samples are greater than 1.5.

The applicant would like to point out that, while the method of the present application has been described in detail by way of the foregoing embodiments, the present application is not limited to the method detailed above; in other words, implementation of the subject matter of the present application does not necessarily depend on the method detailed above. As would be understood by a person skilled in the art, any improvement made to the present application, any equivalent substitution of, and the addition of any auxiliary ingredient into, the raw materials used in the product of the present application, and any specific method chosen to implement the subject matter of the present application shall fall within the scope of the present application and of the patent protection sought by the applicant.

1.-13. (canceled)

14. A method for determining the invasiveness of a bladder cancer, the method comprising:
(a) performing RNA in-situ hybridization on sections of a biological sample comprising bladder cancer cells obtained from a subject and fixed in a neutral buffered formalin on positively charged slides using a first probe that targets an intron sequence of imprinted gene Grb 10 and a second probe that targets an intron sequence of imprinted gene Diras3;
(b) staining the sections with hematoxylin;
(c) microscopically imaging the stained sections and quantifying, for each of the imprinted gene Grb 10 and the imprinted gene Diras3, an expressed quantity of the imprinted gene with a copy number variation, wherein the expressed quantity with the copy number variation is determined by identifying cell nuclei exhibiting more than two red/brown marks after the staining;
(d) calculating a bladder infiltration factor (BIF) as a ratio of the expressed quantity of the imprinted gene Grb 10 with a copy number variation to the expressed quantity of the imprinted gene Diras3 with a copy number variation; and
(e) determining the bladder cancer to be invasive when the BIF is greater than 1.5.

15. The method of claim 14, wherein the RNA in-situ hybridization of step (a) is performed using an RNAscope assay.

16. The method of claim 14, wherein the biological sample is obtained from biopsy cells obtained through cystoscopy or from detached cells in urine of the subject.

17. The method of claim 14, further comprising, prior to step (a), permeabilizing the sections and blocking endogenous peroxidase activity in the sections.

18. The method of claim 14, wherein step (c) further comprises quantifying, for each of the imprinted gene Grb 10 and the imprinted gene Diras3:
(a) a total expressed quantity of the imprinted gene calculated as (b+c+d)/(a+b+c+d)×100%;
(b) an expressed quantity of the imprinted gene being normal calculated as b/(b+c+d)×100%;
(c) an expressed quantity of the imprinted gene with a loss of imprinting calculated as c/(b+c+d)×100%; and
(d) the expressed quantity of the imprinted gene with a copy number variation calculated as d/(b+c+d)×100%;
wherein a is the number of cell nuclei showing no mark, b is the number of cell nuclei showing one red/brown mark, c is the number of cell nuclei showing two red/brown marks, and d is the number of cell nuclei showing more than two red/brown marks.

19. The method of claim 18, wherein the bladder cancer is determined to be non-invasive when:
(a) the total expressed quantity of the imprinted gene Diras3 is not less than 8%,
(b) the expressed quantity of the imprinted gene Diras3 with a copy number variation is greater than 1%,
(c) the total expressed quantity of the imprinted gene Grb 10 is less than 8%, or
(d) the total expressed quantity of the imprinted gene Grb 10 is not less than 8%, the expressed quantity of the imprinted gene Grb 10 with a copy number variation is not greater than 1.5%, and the BIF is less than 1.5.

20. The method of claim 14, wherein the bladder cancer is determined to be a mixed bladder cancer when the total expressed quantity of the imprinted gene Diras3 is not less than 8%, the expressed quantity of the imprinted gene Diras3 with a copy number variation is greater than 1%, the total expressed quantity of the imprinted gene Grb 10 is not less than 8%, the expressed quantity of the imprinted gene Grb10 with a copy number variation is greater than 1.5%, and the BIF is not greater than 1.5.

21. The method of claim 14, wherein the fixing is carried out for at least 24 hours and further comprises embedding the fixed sample in paraffin and cutting the embedded sample into sections.

22. A method for guiding treatment of bladder cancer in a subject, the method comprising:
(a) performing RNA in-situ hybridization on fixed tissue sections of a biological sample comprising bladder cancer cells obtained from a subject using probes targeting intron sequences of imprinted gene Grb 10 and imprinted gene Diras3, respectively;
(b) counterstaining the tissue sections with hematoxylin and microscopically imaging the counterstained tissue sections;
(c) for each of the imprinted gene Grb 10 and the imprinted gene Diras3, determining an expressed quantity of the imprinted gene with a copy number variation based on the number of cell nuclei exhibiting more than two red/brown marks relative to the total number of cell nuclei exhibiting at least one mark;
(d) calculating a bladder infiltration factor (BIF) as a ratio of the expressed quantity of the imprinted gene Grb 10 with a copy number variation to the expressed quantity of the imprinted gene Diras3 with a copy number variation;
(e) classifying the bladder cancer as invasive when the BIF is greater than 1.5, or as non-invasive when the BIF is not greater than 1.5; and
(f) selecting a treatment regimen for the subject based on the classifying, wherein the treatment regimen comprises removal of part or all of the bladder and chemical therapy when the bladder cancer is classified as invasive, or regular cystoscopic examination to remove any recurring tumor via cystoscope when the bladder cancer is classified as non-invasive.

23. The method of claim 22, wherein the RNA in-situ hybridization is performed using an RNAscope assay.

24. The method of claim 22, wherein the biological sample comprises biopsy cells obtained through cystoscopy.

25. The method of claim 22, wherein the probes are designed from intron sequences of the imprinted gene Grb 10 and the imprinted gene Diras3, and the RNA in-situ hybridization further comprises signal amplification.

26. The method of claim 22, wherein the expressed quantity of the imprinted gene with a copy number variation is calculated as d/(b+c+d)×100%, where b is the number of cell nuclei showing one red/brown mark, c is the number of cell nuclei showing two red/brown marks, and d is the number of cell nuclei showing more than two red/brown marks.

27. A method for classifying a bladder cancer as invasive or non-invasive, the method comprising:
(a) performing in-situ hybridization on a tissue section prepared from a bladder cancer sample using a probe specific for an intron sequence of imprinted gene Grb 10 and a probe specific for an intron sequence of imprinted gene Diras3;
(b) detecting hybridization signals in cell nuclei of the tissue section and quantifying, for each of the imprinted gene Grb 10 and the imprinted gene Diras3, a proportion of cell nuclei exhibiting copy number variation signals;
(c) calculating a bladder infiltration factor (BIF) defined as the proportion of cell nuclei exhibiting copy number variation signals for the imprinted gene Grb 10 divided by the proportion of cell nuclei exhibiting copy number variation signals for the imprinted gene Diras3; and
(d) classifying the bladder cancer as invasive when the BIF exceeds a threshold value.

28. The method of claim 27, wherein the threshold value is 1.5.

29. The method of claim 27, wherein the in-situ hybridization is RNA in-situ hybridization and the hybridization signals are detected after counterstaining with hematoxylin.

30. The method of claim 27, wherein a cell nucleus exhibiting copy number variation signals is identified as a cell nucleus showing more than two discrete hybridization signals after counterstaining.

31. The method of claim 27, further comprising selecting a treatment for the bladder cancer based on the classifying of step (d).
